(19) United States
(12) Patent Application Publication
(10) Pub. No.: US 2025/0213285 A1
(43) Pub. Date: Jul. 3, 2025

(54) METHOD AND APPARATUS FOR ABLATING THE VEIN OF MARSHALL

(71) Applicant: Boston Scientific Scimed, Inc., Maple Grove, MN (US)

(72) Inventors: Allan Charles Shuros, St. Paul, MN (US); Eduardo Moriyama, Toronto (CA); Prateek Mathur, Toronto (CA); Carline Quach, Concord (CA)

(21) Appl. No.: 18/991,018

(22) Filed: Dec. 20, 2024

Related U.S. Application Data
(60) Provisional application No. 63/615,075, filed on Dec. 27, 2023.

(51) Int. Cl. A61B 18/00; A61B 90/00

(52) U.S. Cl. CPC A61B 18/00; A61B 90/06; A61B 2018/00083; A61B 2018/00178; A61B 2018/00184; A61B 2018/00351; A61B 2018/00577; A61B 2018/00613; A61B 2018/00839

(57) ABSTRACT

A device for ablating tissue adjacent the vein of Marshall in a patient includes an elongated shaft having a proximal portion including a proximal end and an opposite distal portion including a distal end. The distal portion includes a diameter capable of being inserted into the vein of Marshall. At least one ablation electrode is located on the distal portion of the elongated shaft. A connector is located at the proximal end of the elongated shaft. The connector is configured to removably connect to a control system for delivery of a pulsed field to the at least one ablation electrode. A movable insulation sheath surrounds the elongated shaft and is configured for adjustment of an exposed surface area of the at least one ablation electrode.

### CROSS REFERENCE TO RELATED APPLICATIONS

The present applications claims priority to, and the benefit of, U.S. Provisional Patent Application No. 63/615,075, filed Dec. 27, 2023, the entire disclosure of which is incorporated herein by reference.

### TECHNICAL FIELD

The present disclosure relates to medical systems and methods for ablating tissue in a patient. More specifically, the present disclosure relates to medical systems and methods for ablation of tissue adjacent the vein of Marshall.

### BACKGROUND

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques indiscriminately kill tissue through cell necrosis, which may damage or kill otherwise healthy tissue, such as tissue in the esophagus, phrenic nerve cells, and tissue in the coronary arteries. While thermal ablation techniques are frequency used, non-thermal techniques such as pulsed field ablation can also be used for treating tissue.

Pulsed field ablation involves the application of short pulsed electric fields that may reversibly irreversibly destabilize cell membranes through electropermeabilization. These fields generally do not affect the structural integrity of the tissue components, including the acellular cardiac extracellular matrix. This allows for very brief periods of therapeutic energy delivery, on the order of tens of milliseconds in duration, leading to significantly less collateral damage of non-targeted tissue as associated with thermal ablation techniques.

### SUMMARY

Example 1 is a device for ablating tissue adjacent the vein of Marshall in a patient. The device includes an elongated shaft having a proximal portion including a proximal end and an opposite distal portion including a distal end. The distal portion includes a diameter capable of being inserted into the vein of Marshall. At least one ablation electrode is located on the distal portion of the elongated shaft. A connector is located at the proximal end of the elongated shaft. The connector is configured to removably connect to a control system for delivery of a pulsed field to the at least one ablation electrode. A movable insulation sheath surrounds the elongated shaft and is configured for adjustment of an exposed surface area of the at least one ablation electrode.

Example 2 is the device of Example 1, wherein the diameter is in the range of 0.010″ to 0.1″.

Example 3 is the device of Example 1 or 2, wherein the at least one ablation electrode includes a first ablation electrode located at the distal end, and a second ablation electrode located proximal of the distal end.

Example 4 is the device of Example 3, wherein the at least one ablation electrode includes a third ablation electrode located proximal of the second ablation electrode.

Example 5 is the device of any of Examples 1-4, wherein the exposed surface area of the at least one ablation electrode is in the range of 1.41 mm2 to 28.2 mm2.

Example 6 is the device of any of Examples 1-5, further comprising a magnetic sensor located in the distal portion.

Example 7 is the device of any of Examples 1-6, wherein the distal portion includes a preformed curve configured to allow for steering around tortuous anatomy and into the vein of Marshall.

Example 8 is the device of any of Examples 1-7, further comprising at least one steering wire configured to impart a change in shape of the distal portion.

Example 9 is the device of any of Examples 1-8, further comprising at least one sensing electrode located on the distal portion.

Example 10 is the device of Example 9, wherein the at least one sensing electrode is an EMG electrode.

Example 11 is the device of Example 9, wherein the at least one sensing electrode or the at least one ablation electrode comprises a plurality of evenly spaced electrodes.

Example 12 is the device of Example 9, wherein the at least one sensing electrode or the at least one ablation electrode comprises a plurality of unevenly spaced electrodes.

Example 13 is the device of any of Examples 1-12, further comprising a lumen that extends between the proximal end and the distal end of the elongated shaft.

Example 14 is the device of Example 13, wherein the lumen is configured to receive a fluid.

Example 15 is the device of Example 13, wherein the lumen is configured to accommodate a guidewire.

Example 16 is a device for ablating tissue adjacent the vein of Marshall in a patient. The device includes an elongated shaft having a proximal portion including a proximal end and an opposite distal portion including a distal end. The distal portion includes a preformed curve configured to allow for steering around tortuous anatomy and into the vein of Marshall. A plurality of ablation electrodes are located on the distal portion of the elongated shaft. The plurality of ablation electrodes includes a first ablation electrode located at the distal end of the elongated shaft and a second ablation electrode located proximal of the first ablation electrode. A connector is located at the proximal end of the elongated shaft. The connector is configured to removably connect to a control system for delivery of a pulsed field to the plurality of ablation electrode. A movable insulation sheath surrounds the elongated shaft and is configured for translation over one or more of the plurality of ablation electrodes.

Example 17 is the device of Example 16, further comprising at least one steering wire configured to adjust the shape of the preformed curve.

Example 18 is the device of Example 17, wherein the distal portion includes a diameter in the range of 0.010″ to 0.1″.

Example 19 is the device of Example 16, wherein the at least one ablation electrode includes a third ablation electrode located proximal of the second ablation electrode.

Example 20 is the device of Example 16, wherein the exposed surface area of the first ablation electrode is in the range of 1.41 mm2 to 28.2 mm2.

Example 21 is the device of Example 16, further comprising a magnetic sensor located in the distal portion.

Example 22 is the device of Example 16, further comprising at least one sensing electrode located on the distal portion.

Example 23 is the device of Example 22, wherein the at least one sensing electrode is an EMG electrode.

Example 24 is the device of Example 22, wherein the at least one sensing electrode or the plurality of ablation electrodes comprises a plurality of evenly spaced electrodes.

Example 25 is the device of Example 22, wherein the at least one sensing electrode or the plurality of ablation electrodes comprises a plurality of unevenly spaced electrodes.

Example 26 is the device of Example 16, further comprising a lumen that extends between the proximal end and the distal end of the elongated shaft.

Example 27 is the device of Example 12, wherein the lumen is configured to receive a fluid or to accommodate a guidewire.

Example 28 is a method for ablating tissue adjacent the vein of Marshall in a patient. The method includes providing an ablation device, the ablation device including an elongated shaft having a proximal portion including a proximal end and an opposite distal portion including a distal end. At least one ablation electrode is located on the distal portion of the elongated shaft. A connector is located at the proximal end of the elongated shaft. The connector is configured to removably connect to a control system for delivery of a pulsed field to the at least one ablation electrode. A movable insulation sheath surrounds the elongated shaft and is configured for adjustment of an exposed surface area of the at least one ablation electrode. The method includes advancing the ablation device through the vasculature of the patient to position the distal portion of the elongated shaft within the vein of Marshall. The method includes translating the movable insulation sheath to a desired position and providing a pulsed field to the at least one ablation electrode to ablate tissue adjacent the vein of Marshall.

Example 29 is the method of Example 28, wherein the tissue adjacent the vein of Marshall includes myocardial sleeves and adjacent myocardium.

Example 30 is the method of Example 28, wherein the tissue adjacent the vein of Marshall includes a source of ventricular arrhythmias.

Example 31 is the method of Example 28, wherein the at least one ablation electrode includes first ablation electrode located at the distal end and a second ablation electrode located proximal of the first ablation electrode.

Example 32 is a method for ablating tissue adjacent the vein of Marshall in a patient. The method includes providing an ablation device, the ablation device including an elongated shaft having a proximal portion including a proximal end and an opposite distal portion including a distal end. A plurality of ablation electrodes are located on the distal portion of the elongated shaft. The plurality of ablation electrodes includes a first ablation electrode located at the distal end of the elongated shaft and a second ablation electrode located proximal of the first ablation electrode. A connector is located at the proximal end of the elongated shaft. The connector is configured to removably connect to a control system for delivery of a pulsed field to the plurality of ablation electrodes. The ablation device includes one or more sensor. The method includes using positional data from the one or more sensor and advancing the ablation device through the vasculature of the patient to position the distal portion of the elongated shaft within the vein of Marshall. The method includes providing a pulsed field to the plurality of ablation electrodes to ablate tissue adjacent the vein of Marshall.

Example 33 is the method of Example 32, wherein the tissue adjacent the vein of Marshall includes myocardial sleeves and adjacent myocardium.

Example 34 is the method of Example 32, wherein the tissue adjacent the vein of Marshall includes a source of ventricular arrhythmias.

Example 35 is the method of Example 32, wherein the at least one ablation electrode includes first ablation electrode located at the distal end and a second ablation electrode located proximal of the first ablation electrode.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an ablation device positioned within a vein of Marshall of a patient, in accordance with the disclosure.

FIG. 2 is an illustration of a first embodiment of an ablation device, in accordance with the disclosure.

FIG. 3 is an illustration of a second embodiment of an ablation device, in accordance with the disclosure.

FIG. 4 is an illustration of a third embodiment of an ablation device, in accordance with the disclosure.

FIG. 5 is a flowchart illustrating a method for ablating tissue adjacent the vein of Marshall, in accordance with the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

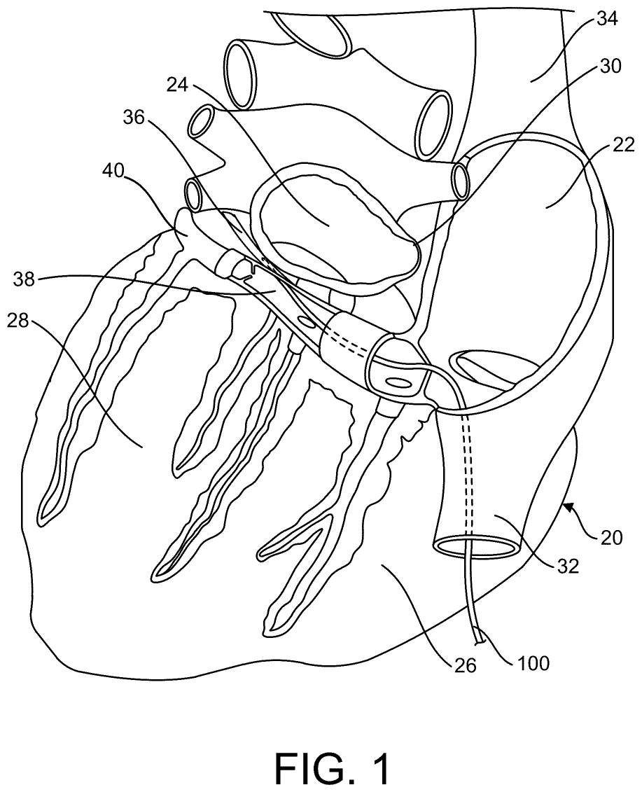

FIG. 1 is a diagram illustrating an ablation device 100 positioned within a vein of Marshall 36 of a patient, in accordance with the disclosure. As is known, the human heart 20 has four chambers, a right atrium 22, a left atrium 24, a right ventricle 26 (not shown in cross-section) and a left ventricle 28 (not shown in cross-section). Separating the right atrium 22 and the left atrium 24 is an atrial septum 30. As is further known, deoxygenated blood from the patient's body is returned to the right atrium 22 via an inferior vena cava (IVC) 32 or a superior vena cava (SVC) 34.

The vein of Marshall 36, also known as the oblique vein of Marshall or the oblique vein of the left atrium 24 is a small vein that descends on and drains the posterior wall of the left atrium 24. It drains directly into the coronary sinus 38 at the same end as the great cardiac vein 40, marking the origin of the sinus 38. The vein of Marshall 36 is located typically at the junction of the coronary sinus 38 and the great cardiac vein 40.

Like pulmonary veins, the vein of Marshall 36 often contains both autonomic innervation and myocardial muscular sleeves that provide interatrial conduction pathways between the right atrium 22 and the left atrium 24 through the coronary sinus 38. Also, these muscular sleeves, similar to pulmonary veins, can be a source of arrhythmogenic foci, contributing to the maintenance of atrial fibrillation (AF). Perimitral flutter atrial tachycardias (AT) often occur following AF ablation. Mitral isthmus ablation is used to stop the AT, but the procedure can be complicated by VOM conduction pathways. As such the VOM is an attractive target of ablation for both AF and AT.

The ablation device 100 is configured to deliver ablative energy to the vein of Marshall 36 and tissue adjacent the vein of Marshall 36 to electrically isolate it from the rest of the heart 20. Tissue adjacent the vein of Marshall 36 can include the vein of Marshall 36 itself and surrounding tissue including but not limited to myocardial sleeves and adjacent myocardium.

The ablation device 100 is configured to enter into and traverse the vein of Marshall 36 to deliver ablative energy to a desired location. While illustrated as being advanced along the IVC 32, the ablation device 100 may also enter the vein of Marshall 36 from the SVC 34. The ablation device 100 can take the form of a guidewire, catheter, or other elongated member having a diameter, stiffness, biocompatibility, and shape suitable for being directed into the vein of Marshall 36.

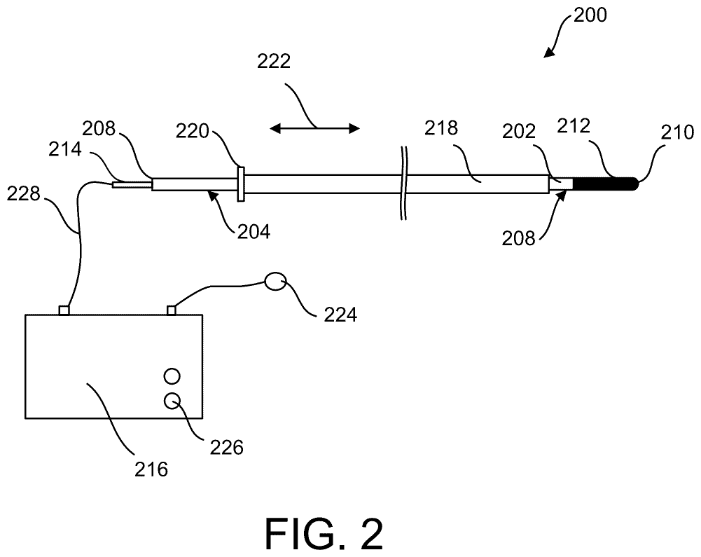

FIG. 2 is an illustration of a first embodiment of an ablation device 200, in accordance with the disclosure. The ablation device 200 is configured for ablating tissue adjacent the vein of Marshall 36 in a patient. The device 200 includes an elongated shaft 202 having a proximal portion 204 including a proximal end 206 and an opposite distal portion 208 including a distal end 210. The distal portion 208 includes a diameter capable of being inserted into the vein of Marshall 36.

At least one ablation electrode 212 is located on the distal portion 208 of the elongated shaft 202. The at least one ablation electrode 212 encompasses the distal end 210 of the elongated shaft 202 and extends proximally. The at least one ablation electrode 212 has an exposed surface area in the range of 1.41 mm² to 28.2 mm². One or more leads or conductors connect the at least one ablation electrode 212 to a connector 214 located at the proximal end 206 of the shaft 204.

The connector 214 is configured to removably connect to a control system 216 for delivery of a pulsed field to the at least one ablation electrode 212 using one or more wire or cable 228. The control system 216 includes input means 226 to modify one or more parameters associated with the pulsed filed, such as frequency, duration, strength, voltage, or other parameters that affect the delivery of energy to tissue. Pulsed field ablation involves the application of short pulsed electric fields (PEF), which may reversibly or irreversibly destabilize cell membranes through electropermeabilization. Pulse field ablation does not affect the structural integrity of the tissue components, including the acellular cardiac extracellular matrix. The nature of PFA allows for very brief periods of therapeutic energy delivery, on the order of tens of milliseconds in duration. The control system 216 can be configured for delivering pulses to the tissue using the at least one ablation electrode 212 and an external return electrode 224.

In some embodiments, the control system 216 can provide voltage pulse waveforms that are hierarchical and have a nested structure. For example, the pulse waveform may include hierarchical groupings of pulses having associated timescales. In some embodiments, the methods, systems, and devices disclosed herein may comprise one or more of the methods, systems, and devices described in International Application Serial No. PCT/US2016/057664, filed on Oct. 19, 2016, and titled “SYSTEMS, APPARATUSES AND METHODS FOR DELIVERY OF ABLATIVE ENERGY TO TISSUE,” the contents of which are hereby incorporated by reference in its entirety.

In other embodiments, the ablation electrode 212 may be an RF electrode having a bipolar configuration, or a monopolar configuration with a dispersive grounding pad on the patient's skin to complete the electrical circuit. Ablation energy may be provided by the control system 216 as radiofrequency electrical current having a frequency up to 1 MHZ, 50 MHz or 100 MHz or in a range of about 300 to 1 MHz or about 300 to 700 KHz and a power in a range of about 1 to 50 W. The delivery of RF energy may be controlled by the control system 216 associated with a controller that uses temperature feedback from a sensor associated with the control system 216.

A movable insulation sheath 218 surrounds the elongated shaft 202 and is configured for adjustment of an exposed surface area of the at least one ablation electrode 212. The movable insulation sheath 218 includes a gripping portion 220, such as a hub, that can be gripped by a user to translate the movable insulation sheath 218 along the length of the elongated shaft 202. Arrow 222 indicates how the movable insulation sheath 218 can be retracted or extended along the elongated shaft 202 in order to modify the exposed portion of the at least one ablation electrode 212. In order to reduce the exposed surface area of the at least one ablation electrode 212, the movable insulation sheath 218 is extended toward the distal end 210 of the elongated shaft 202. Conversely, to increase the exposed surface area of the at least one ablation electrode 212, the movable insulation sheath 218 is retracted toward the proximal end 206 of the elongated shaft 202.

The elongated shaft 202 is formed of a biocompatible material and has a cross-sectional shape suitable for introduction into the vein of Marshall 36. In one aspect, the elongated shaft 202 has a circular cross-section and includes a diameter in the range of 0.010″ to 0.1″ at the distal portion 208. In one aspect, the elongated shaft 202 is solid. In another aspect, the elongated shaft 202 includes a lumen that extends between the proximal end and the distal end. The lumen is configured to receive a fluid, such as a contrasting liquid, or to accommodate a medical device, such as a mandrel, guidewire, or stylet.

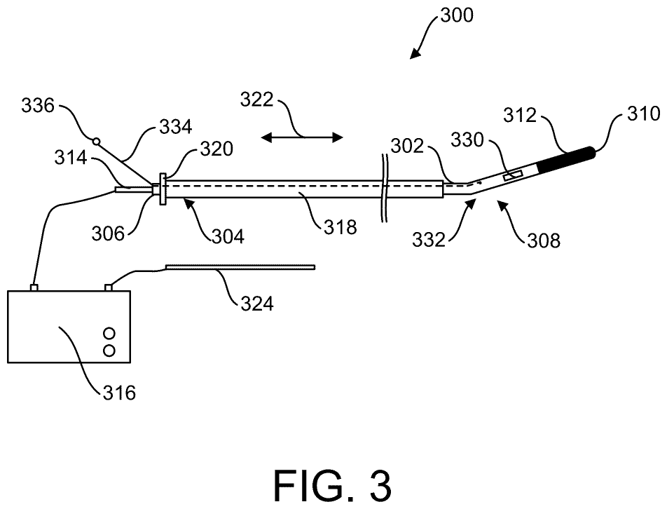

FIG. 3 is an illustration of a second embodiment of an ablation device 300 capable of being inserted into and delivering energy to the vein of Marshall 36, in accordance with the disclosure. Similar to the device 200 of FIG. 2, device 300 includes an elongated shaft 302 having a proximal portion 304 including a proximal end 306 and an opposite distal portion 308 including a distal end 310.

At least one ablation electrode 312 is located on the distal portion 308 of the elongated shaft 302. The at least one ablation electrode 312 encompasses the distal end 310 of the elongated shaft 302 and extends proximally. One or more leads or conductors connect the at least one ablation electrode 312 to a connector 314 located at the proximal end 306 of the shaft 304.

A magnetic sensor 330 is located in the distal portion 308. The magnetic sensor 330 can be electrically connected to a control system 316 by one or more leads or conductors that connect the magnetic sensor 330 to the connector 314. The magnetic sensor 330 can be used by the control system 316 to create a map of internal structures of the heart 20 and can aid in positioning the ablation device 300 within the heart 20. The magnetic sensor 330 is a 5 degrees-of-freedom or a 6 degrees-of-freedom sensor.

In order to aid in navigation through a patient's vascular system and into the vein of Marshall 36, the distal portion 308 of ablation device 300 includes a preformed curve 332. The preformed curve 332 is configured such that the distal portion 308 achieves a non-linear shape in an unconstrained configuration. In one aspect, the preformed curve 332 angles the distal portion 308 away from a longitudinal axis of the elongated shaft 302. The preformed curve 332 may offset the distal portion 308 with respect to the longitudinal axis at an angle in the range of 1 degree to 35 degrees.

In some aspects, the preformed curve 332 may be configured such that the distal portion 308 achieves a shape having one or more curves in an unconstrained state. For example, the preformed curve 332 may include a J-shape, S-shape, or spiral shape.

The ablation device 300 includes at least one steering wire 334 that passes through at least a portion of the elongated shaft 302. The at least one steering wire 334 is affixed to the distal portion 308 of the elongate shaft 302 and is configured to impart a change in shape of the distal portion 308. The at least one steering wire 334 may be actuated by grasping a pull ring 336 and applying tension to the at least one steering wire 334. In one aspect, the at least one steering wire 334 can be configured such that pulling the at least one steering wire 334 further deflects the distal portion 308 away from the longitudinal axis. In another aspect, the at least one steering wire 334 can be configured such that pulling the at least one steering wire 334 pulls the distal portion 308 towards the longitudinal axis.

A movable insulation sheath 318 surrounds the elongated shaft 302 and is configured for adjustment of an exposed surface area of the at least one ablation electrode 312. The movable insulation sheath 318 includes a gripping portion 320, such as a hub, that can be gripped by a user to translate the movable insulation sheath 318 along the length of the elongated shaft 302. Arrow 322 indicates how the movable insulation sheath 318 can be retracted or extended along the elongated shaft 302 in order to modify the exposed portion of the at least one ablation electrode 312.

The control system 316 can be configured for delivering pulses to the tissue using the at least one ablation electrode 312 and an external return electrode as discussed above or an internal electrode in the form of a wire or catheter 324. Using an internal electrode allows for precise delivery across structures within the body, such as a cardiac wall.

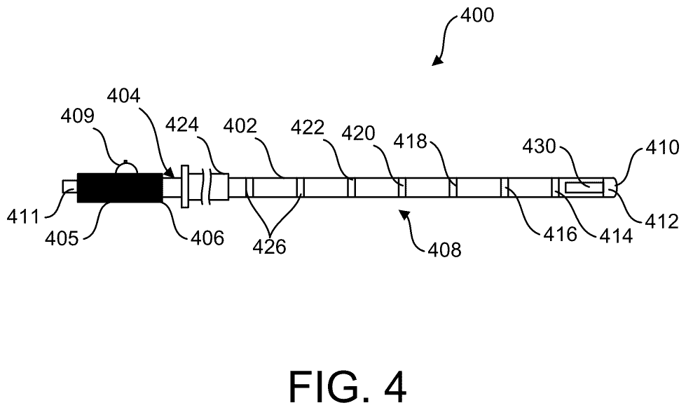

FIG. 4 is an illustration of a third embodiment of an ablation device 400 capable of being inserted into and delivering energy to the vein of Marshall 36, in accordance with the disclosure. Similar to ablation device 200 of FIG. 2 and ablation device 300 of FIG. 3, ablation device 400 includes an elongated shaft 402 having a proximal portion 404 including a proximal end 406 and an opposite distal portion 408 including a distal end 410.

A handle 407 is connected to the proximal end 406 and includes a steering knob 409. The steering knob 409 is actuated by a user to change the shape of the distal portion 408 of the elongated shaft 402. One or more steering wires connect the steering knob 409 to the distal portion 408 and apply tension to the distal portion 408 to change the shape upon actuation of the steering knob 409. The handle 407 includes a connector 411 configured to electrically couple components of the ablation device 400 to a control system (not shown) as discussed previously. The control system is configured to control energy delivered to tissue as well as monitoring any sensors associated with ablation device 400.

A plurality of ablation electrodes 412, 414, 416, 418, 420, 422 are located along the distal portion 408 of the elongated shaft 402. The plurality of ablation electrode 412, 414, 416, 418, 420, 422 includes at least a first ablation electrode 412 located at the distal end 410, a second ablation electrode 414 located proximal of the distal end 410, a third ablation electrode 416 located proximal of the second ablation electrode 414, a fourth ablation electrode 418 located proximal of the third ablation electrode 416, a fifth ablation electrode 420 located proximal of the fourth ablation electrode 418, and a sixth ablation electrode 422 located proximal of the fifth ablation electrode 420. In one aspect, the plurality of ablation electrodes 412, 414, 416, 418, 420, 422 are evenly spaced along the distal portion 408 of the elongated shaft 402. In another aspect, the plurality of ablation electrodes 412, 414, 416, 418, 420, 422 are unevenly spaced along the distal portion 408 of the elongated shaft 402. In some aspects, the plurality of ablation electrodes 412, 414, 416, 418, 420, 422 share the same length. In other aspects, the plurality of ablation electrodes 412, 414, 416, 418, 420, 422 have different lengths.

In one aspect, the control system selects one or more of the plurality of ablation electrodes 412, 414, 416, 418, 420, 422 to deliver energy to a desired tissue. The energy can include a pulse field in a first configuration. In another configuration, the energy can include thermal energy, for example radiofrequency energy. The control system is configured to deliver a first type of energy to at least one of the plurality of ablation electrodes 412, 414, 416, 418, 420, 422 and to deliver a second type of energy to at least one other of the plurality of ablation electrodes 412, 414, 416, 418, 420, 422. In another aspect, a movable insulation sheath 424 can be used to expose or cover one or more of the plurality of ablation electrodes 412, 414, 416, 418, 420, 422 as desired to increase or limit a treatment area.

The distal portion 408 of the elongated shaft 402 also includes at least one sensing electrode 426 and a magnetic sensor 430 as discussed previously. The movable sheath 424 can be translated to expose one or more of the at least one sensing electrode 426. In one aspect, the at least one sensing electrode 426 includes a plurality of sensing electrodes. The sensing electrodes are configured to measure one or more parameter to aid in the placement of the ablation device 400 at a desired location, monitor the ablation procedure, or measure a result of the ablation procedure. For example, the at least one sensing electrode 426 can be configured as a temperature measuring electrode, pacing electrode, an EMG electrode, EKG electrode, position sensing electrode, or other sensing electrode. In one aspect, the sensing electrodes 426 are evenly spaced along the distal portion 408 of the elongated shaft 402. In another aspect, the sensing electrodes 426 are unevenly spaced along the distal portion 408 of the elongated shaft 402.

One or more leads or conductors connect the plurality of ablation electrodes 412 and the at least one sensing electrode 426 to the connector 411 on the handle 407. The one or more leads or conductors are poisoned within the ablation device 400 in a lumen or channel therein.

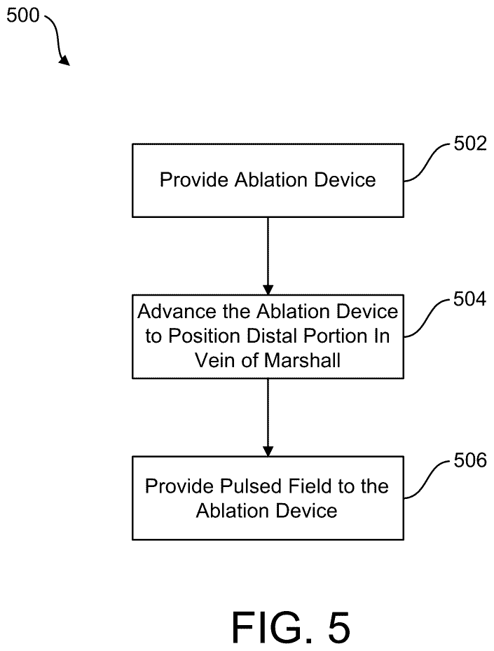

FIG. 5 is a flowchart illustrating a method 500 for ablating tissue adjacent the vein of Marshall, in accordance with the disclosure. Step 502 includes providing an ablation device. The ablation device can be an ablation device as described above in FIGS. 2-4. Step 504 includes advancing the ablation device through the vasculature of the patient to position the distal portion of the elongated shaft within the vein of Marshall. Advancing the ablation device can include using positional data from one or more sensor associated with the ablation device. For example, positional data and electrograms may be collected and displayed on the control system or an electroanatomical mapping system. Additionally, supplementary devices, such as a guide sheath, balloon catheter, or guidewire, may be used to help guide the ablation device through the coronary sinus and into the vein of Marshall. The ablation device is positioned in such a manner such that one or more ablation electrode is adjacent desired tissue to be ablated. Once positioned, step 506 includes providing a pulsed field to the at least one ablation electrode to ablate tissue adjacent the vein of Marshall. The tissue can include myocardial sleeves and adjacent myocardium. Following ablation of tissue adjacent the vein of Marshall, the ablation device can be navigated to another location to ablate other desired tissue, for example, the ablation device can be navigated to an epicardial coronary artery for targeting a source of ventricular arrhythmias.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A device for ablating tissue adjacent the vein of Marshall in a patient, the device comprising:
an elongated shaft having a proximal portion including a proximal end and an opposite distal portion including a distal end, the distal portion including a preformed curve configured to allow for steering around tortuous anatomy and into the vein of Marshall;
a plurality of ablation electrodes located on the distal portion of the elongated shaft, wherein the plurality of ablation electrodes includes a first ablation electrode located at the distal end of the elongated shaft and a second ablation electrode located proximal of the first ablation electrode;
a connector located at the proximal end of the elongated shaft, the connector being configured to removably connect to a control system for delivery of a pulsed field to the plurality of ablation electrodes; and
a movable insulation sheath surrounding the elongated shaft, the movable insulation sheath being configured for translation over one or more of the plurality of ablation electrodes.

2. The device of claim 1, further comprising at least one steering wire configured to adjust the shape of the preformed curve.

3. The device of claim 2, wherein the distal portion includes a diameter in the range of 0.010″ to 0.1″.

4. The device of claim 1, wherein the at least one ablation electrode includes a third ablation electrode located proximal of the second ablation electrode.

5. The device of claim 1, wherein the exposed surface area of the first ablation electrode is in the range of 1.41 mm² to 28.2 mm².

6. The device of claim 1, further comprising a magnetic sensor located in the distal portion.

7. The device of claim 1, further comprising at least one sensing electrode located on the distal portion.

8. The device of claim 7, wherein the at least one sensing electrode is an EMG electrode.

9. The device of claim 7, wherein the at least one sensing electrode or the plurality of ablation electrodes comprises a plurality of evenly spaced electrodes.

10. The device of claim 7, wherein the at least one sensing electrode or the plurality of ablation electrodes comprises a plurality of unevenly spaced electrodes.

11. The device of claim 1, further comprising a lumen that extends between the proximal end and the distal end of the elongated shaft.

12. The device of claim 11, wherein the lumen is configured to receive a fluid or to accommodate a guidewire.

13. A method for ablating tissue adjacent the vein of Marshall in a patient, the method comprising:
providing an ablation device, the ablation device comprising:
an elongated shaft having a proximal portion including a proximal end and an opposite distal portion including a distal end;
at least one ablation electrode located on the distal portion of the elongated shaft;
a connector located at the proximal end of the elongated shaft, the connector being configured to removably connect to a control system for delivery of a pulsed field to the at least one ablation electrode; and
a movable insulation sheath surrounding the elongated shaft, the movable insulation sheath being configured for adjustment of an exposed surface area of the at least one ablation electrode;
advancing the ablation device through the vasculature of the patient to position the distal portion of the elongated shaft within the vein of Marshall;
translating the movable insulation sheath to a desired position; and
providing a pulsed field to the at least one ablation electrode to ablate tissue adjacent the vein of Marshall.

14. The method of claim 13, wherein the tissue adjacent the vein of Marshall includes myocardial sleeves and adjacent myocardium.

15. The method of claim 13, wherein the tissue adjacent the vein of Marshall includes a source of ventricular arrhythmias.

16. The method of claim 13, wherein the at least one ablation electrode includes first ablation electrode located at the distal end and a second ablation electrode located proximal of the first ablation electrode.

17. A method for ablating tissue adjacent the vein of Marshall in a patient, the method comprising:
providing an ablation device, the ablation device comprising:
an elongated shaft having a proximal portion including a proximal end and an opposite distal portion including a distal end;
a plurality of ablation electrodes located on the distal portion of the elongated shaft, wherein the plurality of ablation electrodes includes a first ablation electrode located at the distal end of the elongated shaft and a second ablation electrode located proximal of the first ablation electrode;
a connector located at the proximal end of the elongated shaft, the connector being configured to removably connect to a control system for delivery of a pulsed field to the plurality of ablation electrodes; and
one or more sensor;
using positional data from the one or more sensor, advancing the ablation device through the vasculature of the patient to position the distal portion of the elongated shaft within the vein of Marshall; and
providing a pulsed field to the plurality of ablation electrodes to ablate tissue adjacent the vein of Marshall.

18. The method of claim 17, wherein the tissue adjacent the vein of Marshall includes myocardial sleeves and adjacent myocardium.

19. The method of claim 17, wherein the tissue adjacent the vein of Marshall includes a source of ventricular arrhythmias.

20. The method of claim 17, wherein the at least one ablation electrode includes first ablation electrode located at the distal end and a second ablation electrode located proximal of the first ablation electrode.
